(19) United States
(12) Patent Application Publication
(10) Pub. No.: US 2024/0131226 A1
(43) Pub. Date: Apr. 25, 2024

(54) Porous Osteoinductive Composites

(71) Applicants: Kuros Biosciences B.V., Bilthoven (NL); Medskin Solutions Dr. Suwelack AG, Billerbeck (DE)

(72) Inventors: Florence De Groot-Barrere, Utrecht (NL); Nathan Kucko, Arnhem (NL); Charlie Campion, Amsterdam (NL); Huipin Yuan, Zeist (NL); Kay Mohr, Südlohn (DE); Wolfgang Ernst Weinmar, Rosendahl (DE); Annalena Anja Völker, Nottuln (DE); Claudia Doberenz, Billerbeck (DE)

(21) Appl. No.: 18/275,263

(22) PCT Filed: Feb. 2, 2022
(86) PCT No.: PCT/NL2022/050049

§ 371 Date: Jul. 31, 2023

(30) Foreign Application Priority Data
Feb. 2, 2021 (NL) 2027479

(51) Int. Cl. A61L 27/46; A61L 27/24; A61L 27/56

(52) U.S. Cl. CPC A61L 27/46; A61L 27/24; A61L 27/56; A61L 2430/02

(57) ABSTRACT

The invention is directed to a porous osteoinductive composite comprising osteoinductive granules embedded in a porous matrix, wherein more than 5% of the surface area of the osteoinductive granules is exposed from said matrix as determined by scanning electron microscopy (SEM) imaging. Such a composite can suitably be used in a medical treatment, for example in a medical treatment of connective tissue and/or bone loss or defect.

The invention is directed to osteoinductive composites. In particular, the invention relates to osteoinductive composites comprising osteoinductive granules embedded in a matrix that can be used for the treatment of lost bone or bone defects.

Lost bone or bone defects can be treated by bone graft composite materials. In the art, several of these composite materials are known. For example, osteoconductive composite materials based on calcium-phosphate and a silk or collagen matrix such as those commercially available under the tradenames i-Factor™, Vitoss™, Formagraft™ and Mastergraft™ are known. A drawback of these materials is that they are only osteoconductive, meaning that they lack the possibility to stimulate new bone formation at sites where there is no native bone present to provide osteoblasts.

EP2749301 describes a biocompatible, resorbable composite for osteosynthesis that includes osteoconductive particles dispersed within a porous polymer matrix having a plurality of fluid passageways that expose at least a portion of a plurality of the osteoconductive particles to an exterior of the polymer matrix.

EP2730295 describes a particular calcium phosphate-collagen fiber composite which can induce bone replacement by bone remodeling.

US 2008/0138381 describes a particular bone implant composite comprising a collagen matrix and a calcium-based mineral.

US 2017/0304502 describes a method or making an osteoimplant, which method includes applying a mechanical force to an aqueous slurry of insoluble collagen fibers to entangle the insoluble collagen fibers so as to form a semi-solid mass of entangled insoluble collagen fibers; and lyophilizing the semi-solid mass of entangled collagen fibers to form the osteoimplant. An osteoimplant containing entangled insoluble collagen fibers is also described.

U.S. Pat. No. 5,338,772 describes an implant material which is based on a composite material of calcium phosphate ceramic particles and bioabsorbable polymer.

Composites that induce and stimulate new bone formation in the absence of native bone (i.e. osteoinductive composites) are also known. Osteoinductive composites typically derive their osteoinductivity from osteoinductive granules present in the composite. Examples of synthetic osteoinductive granules include calcium phosphate (CaP) based ceramics as described in WO 2015/009154 (which is incorporated herein in their entirety). An example of a composite including such granules is a putty composite wherein these granules are combined with a polymeric carrier as described in WO 2016/144182 and commercially available as MagnetOs™. A drawback of this putty is however, that it is not porous and does not or very limited allow a clinical practitioner to augment the putty-based construct with autologous tissue of a patient (e.g. blood, bone marrow aspirate, BMA) before the construct is placed in the patient.

It is accordingly desired to provide an osteoinductive composite that does not suffer from the drawbacks of known synthetic bone graft composite materials, and which preferably is porous and/or absorbent and allows to augment the composite with autologous tissue and/or other materials.

The present inventors surprisingly found a correlation between osteoinductive properties and surface exposure of the granules in the composite and that good osteoinductive properties in porous structures can be achieved if the surface of the granules is sufficiently exposed and not covered by the carrier material or matrix that bind the granules in the composite.

Accordingly, the present invention is directed to a porous osteoinductive composite comprising osteoinductive granules comprised in a porous matrix, wherein preferably more than 5% of the surface area of the osteoinductive granules is exposed from said matrix as determined by scanning electron microscopy (SEM) imaging. Sufficient exposure of the surface of the granules can alternatively or additionally be described and/or achieved in other ways as described herein.

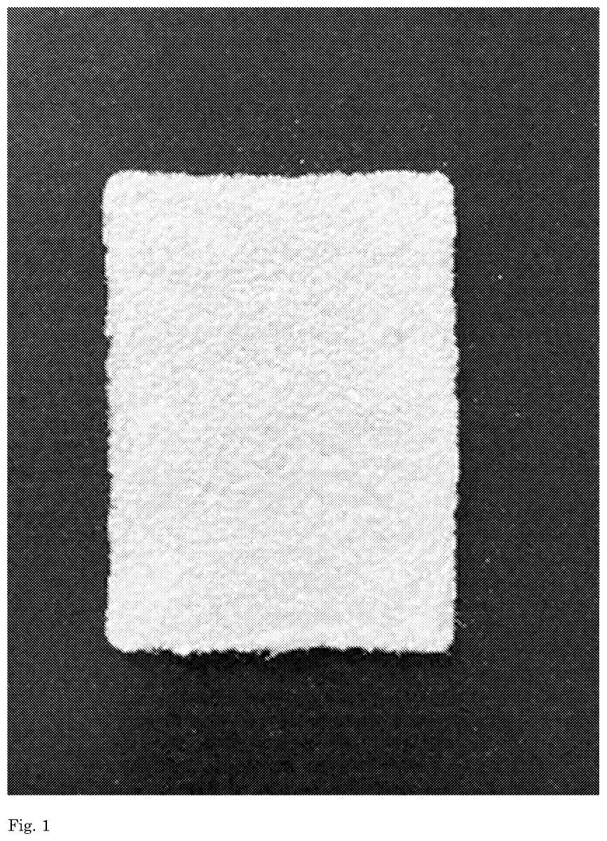

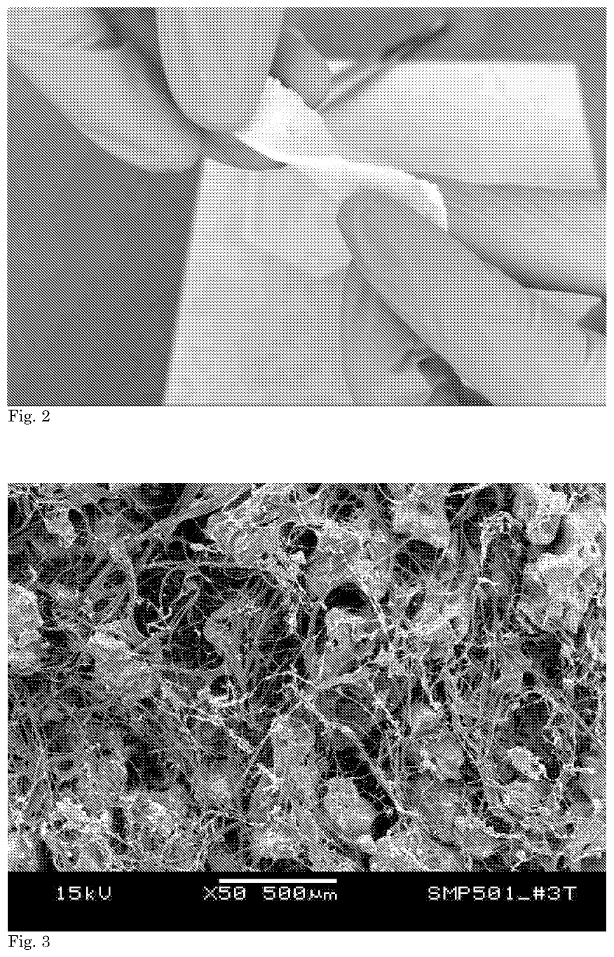

FIGS. 1 and 2 are pictures of a preferred embodiment according to the invention.

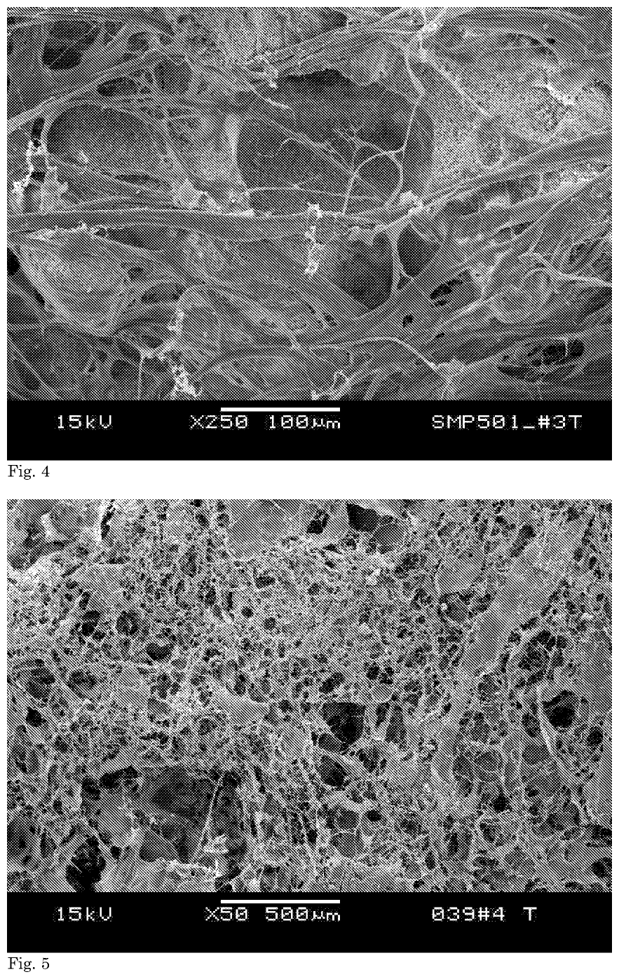

FIGS. 3 and 4 show SEM images of a preferred embodiment according to the invention.

FIGS. 5-8 show SEM images of comparative examples.

The granules according to the present invention are comprised in the matrix, meaning that they are at least partially embedded such as incorporated, fixated and/or surrounded by the matrix. The matrix can be seen as providing a supporting or carrier structure for the granules. However, the matrix does not cover the entire surface of the granules, in contrast as for example the polymer material in a putty as described in WO 2016/144182 does.

Without wishing to be bound by theory, the inventors believe that by providing the surface exposure, a matrix can be used that remains structurally intact under physiological conditions for a sufficiently long period in order for e.g. macrophages, osteoclasts, mesenchymal stem cells (MSc's), osteoblasts and/or osteoprogenitor cells that promote the pro-healing mechanism and/or bone regeneration to invade the composite, while not prohibiting differentiation of the cells to form new bone.

A surface being exposed from the matrix herein means that the surface is not directly covered by the matrix such that the surface is free and accessible for i.a. macrophages, osteoclasts, the osteoblasts and osteoprogenitor cells to contact the surface. From an SEM image, the person skilled in the art can visually differentiate the matrix from granular material and can determine whether a surface area of a granule is exposed or covered by the matrix material.

The surface exposure of the granules in the composite can accordingly be determined by SEM as follows. For a number of representative granules on the SEM image (e.g. from at least three granules), the surface of the granules is analyzed and the amount of said surface covered and exposed is measured. This measurement can be assisted by a software package such as ImageJ.

The inventors found that more surface exposure of the granules leads to more bone formation after implantation of the composite. Accordingly, preferably more than 10%, more preferably more than 20% even more preferably more than 30%, most preferably more than 40% of the surface area of the osteoinductive granules is exposed from said matrix as determined by scanning electron microscopy (SEM) imaging.

The exposure of the surface of the granules can also be expressed as the relative number of granules that exhibit at least a partially exposed surface. Also this can be determined by SEM. For a number of representative granules on the SEM image (e.g. from at least three granules), the surface of the granules is analyzed and the amount of granules exhibiting an at least partially exposed surface is divided by the total amount of detectable granules (either covered or at least partially exposed) visible in the SEM image. In a preferred embodiment, more than 20%, preferably more than 50%, most preferably more that 75% of the granules exhibit an at least partially exposed surface area as determined by scanning electron microscopy (SEM) imaging.

Another factor that can influence the osteoinductive capacity is the morphological structure of the matrix at a micrometer scale (i.e. at a scale of 1-1000 μm). The morphological structure of the matrix can be described by the shape or morphology of its structural elements. Such elements may for example have a bulky shape, or the shape of sheets or fibers at the micrometer scale. With bulky shapes are meant shapes that substantially equally extend in all three dimensions at the micrometer scale. With sheets are meant shapes that substantially equally extend in two dimensions at the micrometer scale. With fibers are meant shapes that extent more in one dimension than in the other two dimensions at the micrometer scale. From SEM images, the person skilled in the art can accordingly describe the structural elements of the matrix at a micrometer scale. Unless explicitly indicated otherwise, the morphology of the matrix and its structural elements is herein described at the micrometer scale.

For good osteoinductivity, it is preferred to that the matrix comprises mostly fibers. The presence of sheets is less preferred and bulky shapes are even less preferred as these elements can obstruct both the surface of the granules and passages into the inner part of the composite. Therefore, in a preferred embodiment, the matrix comprises fibers and sheets in a ratio of more than 1:1, preferably more than 3:1, more preferably more than 4:1, most preferably more than 8:1, wherein fibers are defined as structures having a thickness and width of less than 50 μm and sheets are defined as structure having a thickness and/or width of more than 50 μm. It is accordingly further preferred that the matrix is free from bulky shapes (defined for this purpose as shapes having a size of more than 50 μm in all three dimensions). The morphology of the matrix can thus also be described based on the relative amount of sheet and fiber structures vis-à-vis all structural elements of the matrix. Accordingly, in a preferred embodiment, the matrix comprises more than 50%, preferably more than 70%, more preferably more than 90% fibers and/or less than 50%, preferably less than 30%, more preferably less than 10% sheets.

In a preferred embodiment of the present invention, the matrix is fibrous, meaning that it comprises fibers. Preferably, said fibers have an average diameter of less than 50 μm, preferably less than 30 μm. The length of the fibers may be much longer, e.g. more than 100 μm, or even longer such as more than 500 μm. The diameter of the fibers can be measured using SEM images, optionally assisted by a software package such as ImageJ.

To allow sufficient penetration and access of i.a. macrophages, osteoclasts, the osteoblasts and osteoprogenitor cells into the composite, it preferably exhibits porosity in the range of 60 to 95%, preferably in the range of 70 to 90%. The porosity is herein expressed as the relative voids volume with respect to the total volume of the composite including said voids and can be determined by considering the volume of a sample, measuring the weights of the constituents therein and accordingly calculating the volume of these constituents based on their known densities. The weight and amount of the composite constituents are typically known from its production process. Alternatively, it can be determined as described herein-below for the determination of the weight of matrix and granules.

The porous nature of the composite according to the present invention advantageously allows to uptake of fluids such as autologous tissue fluids. For a facile fluid uptake, the composite preferably exhibits capillary or wicking properties. More preferably, the composite exhibits wicking of more than 50%, preferably more than 100%. Wicking can be determined by immersing the composite in a fluid for a set time point (i.e. 20 sec) and measuring the difference in weight before and after immersion.

The material for the matrix is biocompatible and may be biodegradable. Suitable materials can appropriately be selected by considering material properties such as strength, biodegradability, and capacity to form a porous matrix. As described herein-above, the matrix preferably remains structurally intact in vivo and in vitro under physiological conditions for a sufficiently long period to mitigate migration of the composite in the body. The presence of the matrix material can contribute to this mitigation. Accordingly, the matrix is preferably biodegradation to such an extent that the composite retains matrix material in vivo for at least 24 hours, the time in which the composite can be particularly prone to migration. More preferably, the matrix is preferably resistant to biodegradation to such an extent that the composite retains matrix material in vivo for at least 1 week, preferably for at least 3 weeks. In particular embodiments, the matrix is biodegradation to such an extent that the composite has lost its matrix material in vivo after about 6 weeks or more.

The biodegradability of the composite can also be expressed as the capability of the composite to remain structurally intact. In preferred embodiment, the composite exhibits structural integrity for at least 5 days, preferably at least 12 days under phosphate-buffered saline solution at 37° C. Herein structurally intact means that the composite has a solid shape and its dimensions can be accurately measured.

From other or similar tissue regeneration arts, several polymers and fibrillation technologies are known. Examples of fiber forming methods include electrospinning, solution-blowing, additive manufacturing, lyophilization and the like. See for example Kumar et al., Fibers 6 (2018) 45 (doi:10.3390/fib6030045) and Ligon et al., Chemical Reviews, 117 (2017) 10212-10290 (10.1021/acs.chemrev.7b00074).

Examples of suitable materials for the matrix include natural polymers, semi-synthetic polymers, and synthetic polymers. Accordingly, in a preferred embodiment the matrix comprises:
one or more of natural polymers selected from the group consisting of collagen, gelatin, fibrin, hyaluronic acid, silk fibroin, chitosan, alginate, cellulose, lignin, hydrogels derived from decellularized tissues, and other ECM-derived or ECM-mimicking natural polymers;
one or more semi-synthetic polymers such as gelatin methacryloyl (gelMA), hyaluronic acid methacrylate (HAMA);
one or more synthetic polymers selected from the groups consisting of polyethylene glycol (PEG), polyoxamers, polylactic acid (PLA) such as poly(L-lactic) acid (PLLA), poly(ethylene glycol-co-lactic acid) (PELA), poly(poloxamer-co-lactic acid) (POLA), polyglycolic acid (PGA), poly(lactic-co-glycolic acid) (PGLA), polycaprolactone (PCL), polyvinyl alcohol (PVA), polyamides (PA), polyacrylonitrile (PAN), combinations and co-polymers thereof;
or combinations thereof. See for example Kumar et al., Fibers 6 (2018) 45 (doi:10.3390/fib6030045) and references therein.

Collagen was found to be particularly suitable and preferred as it was found to provide the desired balance between hydrophilicity, strength, biodegradability, flexibility, and morphology options. Type I and type II collagen are particularly preferred. The origin of the collagen may be porcine, bovine, equine, fish and the like. Most preferably, the matrix comprises bovine collagen type I. The collagen may be non-native (e.g. chemically cross-linked) or native. Preferably, the matrix comprises native collagen as this was found to provide the most favorable morphology as described herein.

Native collagen was found to be particularly fibrous. The skilled person can visually differentiate native collagen from non-native collagen using for example SEM images. For instance, the collagen fibril diameter of isolated native collagen is the same or at least approximates the fibril diameter of dermis tissue (e.g. about 90 nm). See for example Dill and Mörgelin, Int Wound J. 17 (2020) 618-630. Also, native collagen is typically more fibrous than non-native collagen.

The granules as used in the composite of the present invention are osteoinductive. This means that the biocompatible composite material comprises one or more material that are osteoinductive. Osteoinductive properties of the biocompatible composite material may for instance be achieved by addition of bone morphogenetic proteins (BMP) and/or other growth factors. In an even further preferred embodiment, the granular synthetic material is intrinsically osteoinductive. This means that the granular synthetic material itself stimulates new bone even in non-osseous environments (e.g. osteoinductive ceramics, demineralized bone matrix-DBM).

WO 2015/009154, which is incorporated herein in its entirety, describes granules based on calcium phosphate CaP that are osteoinductive. It was found that the biodegradable polymeric material of the present invention is suitable to maintain the osteoinductive properties of the granules as described in WO 2015/009154.

The granular synthetic material in accordance with the present invention may comprise calcium phosphate, bioactive glass and the like. Rahaman et al. Acta Biomateralia 2011(6)2355-2373 reviews a variety of synthetic materials for tissue-engineering that are suitable for the present invention. The granules may be ceramic granules. Preferably, the granules comprise calcium phosphate. Such granules have proven to be particularly suitable for tissue regeneration.

The osteoinductive granules preferably have a size in the range of 100 to 2500 μm, preferably 250-1000 μm.

The osteoinductive properties of certain granular synthetic materials are commonly attributed to their specific micro- and sub-microsurface structure. Water may affect this structure and thereby affect the osteoinductive properties of the granular synthetic material. Also other properties of the granular synthetic material may be affected by the presence of water because the synthetic materials may be for instance partially dissolved by the water. Moreover, it is known that water may also affect the osteoinductive potential of other agents, i.e. BMPs and DBM. Therefore, the composite is preferably anhydrous (i.e. comprises less than 2 wt % water based on the total weight of the composite) to prevent or limit loss of the osteoinductive properties. Anhydrous in the present invention therefore means that the environment granular synthetic material is sufficiently anhydrous for the granular synthetic material to sufficiently retain the chemical and structural properties to limit reduction of the biological activity of the granular synthetic material such that the biocompatible composite material remains effective and thus applicable.

The amount of the osteoinductive granules in the composite is preferably more than 50 wt %, more preferably more than 75 wt %, most preferably more than 90 wt % and/or the amount of the matrix in the composite is preferably less than 50 wt %, more preferably less than 25 wt %, most preferably less than 10 wt % based on the weight of the composite. The weight and amount of the composite constituents are typically known from its production process. Alternatively, the weight and amount of the composite constituents can in appropriate embodiments be determined by measuring the ash content of the composite. As such, the ash content reflects the granular inorganic content, while the burned and evaporated organic content reflects the matrix.

The osteoinductive composite according to present invention can have the form of a sheet, strip, block, rod or stick, depending on its intended site for implantation. See FIG. 1 for an exemplary and preferred strip shape. Preferably, it is shapable such as pliable and or flexible and can be cut at 15° C. (i.e. the typical temperature in operatory rooms) as this facilitate placement by the clinical practitioner (see FIG. 2). For good handling, it is further preferred that the composite exhibits a tensile strength of at least 0.1 MPa, preferably 0.1 MPa to 5 MPa and/or an elastic modulus in the range of 2 to 300 MPa.

Another aspect of the present invention is a method for the preparation of the porous osteoinductive composite according to any of the previous claims, said method comprising mixing the osteoinductive granules with a solution comprising the matrix, followed by lyophilizing said mixture.

The osteoinductive composite according to the invention can be use in a medical treatment such as in a medical treatment of connective tissue and/or bone loss or defect.

The osteoinductive composite can induce and guide the three-dimensional regeneration of bone in the defect site into which it is implanted. When placed next to viable host bone, new bone will be deposited on the surface of the implant. The composite resorbs and is replaced by bone during the natural process of bone remodelling.

In a preferred embodiment, the osteoinductive composite is gamma sterilised.

The osteoinductive composite according to the invention can be use as bone void filler for voids and gaps that are not intrinsic to the stability of the bony structure. The osteoinductive composite can be for use in the treatment of surgically created osseous defects or osseous defects resulting from traumatic injury to the bone. The osteoinductive composite can be packed into bony voids or gaps of the skeletal system (i.e. extremities, spine, cranial, mandible, maxilla and pelvis) and may be combined with autogenous bone, blood, platelet rich plasma (PRP) and/or bone marrow.

In particular embodiments, the osteoinductive composite can be used in a medical treatment to replace or supplement autogenous and/or allogenous spongiosa, e.g. in filling and bridging of skeletal bone defects including spine, plastic reconstruction of damaged or resectioned bone areas, filling of intervertebral implants.

In particular embodiments, the osteoinductive composite can be used in a medical treatment to fill or reconstruct multiple walled (artificial or degenerative) bone defects, e.g. defects after the extirpation of bone cyst, augmentation of an atrophied alveolar ridge, sinus lift or sinus floor elevation, filling of alveolar defects after tooth extraction for preservation of the alveolar ridge, filling of extraction defects for creating an implant bed, filling of two- or multiple-walled bone pockets as well as the bi- and trifurcations of teeth, defects after operative removal of retained teeth or corrective osteotomies, other multiple-walled bone defects of the alveolar processes and the facial skull.

Accordingly, another aspect of the present invention is a method for the treatment of connective tissue and/or bone loss or defect of a patient, said method comprising providing the porous osteoinductive composite according to the present invention, optionally augmenting said composite with autologous tissue such as BMA, the blood or PRP of said patient, and implanting the composite in the patient.

As used herein, the singular forms “a”, “an” and “the” are intended to include the plural forms as well, unless the context clearly indicates otherwise. The term “and/or” includes any and all combinations of one or more of the associated listed items. It will be understood that the terms “comprises” and/or “comprising” specify the presence of stated features but do not preclude the presence or addition of one or more other features.

For the purpose of clarity and a concise description features are described herein as part of the same or separate embodiments, however, it will be appreciated that the scope of the invention may include embodiments having combinations of all or some of the features described.

The present invention can be illustrated by the following non-limiting examples.

Preparation and Analysis of Composite Strips

Composites were prepared by mixing MagnetOs™ Granules 250-1000 μm in size with different aqueous collagen solutions at a ratio of 0.7 ml granules per 1.0 ml of sample material. The mixture was then poured into a stainless-steel mold, frozen, subjected to lyophilization and sterilized using gamma-irradiation before performing physicochemical characterization testing and in vivo evaluation.

Table 1 show the physical and chemical properties of the composite strips that were obtained. SEM images are provided in FIGS. 3-9.

For Example 1, native bovine type 1 collagen provided by MedSkin Solutions Dr. Suwelack A G, Germany was used. SEM images for Example 1 are provided in FIGS. 3 and 4.

For Comparative Example 1-10, bovine type I collagen supplied and processed by SouthernLights Biomaterials was used.

FIG. 5 shows a SEM image of Comparative Example 1.

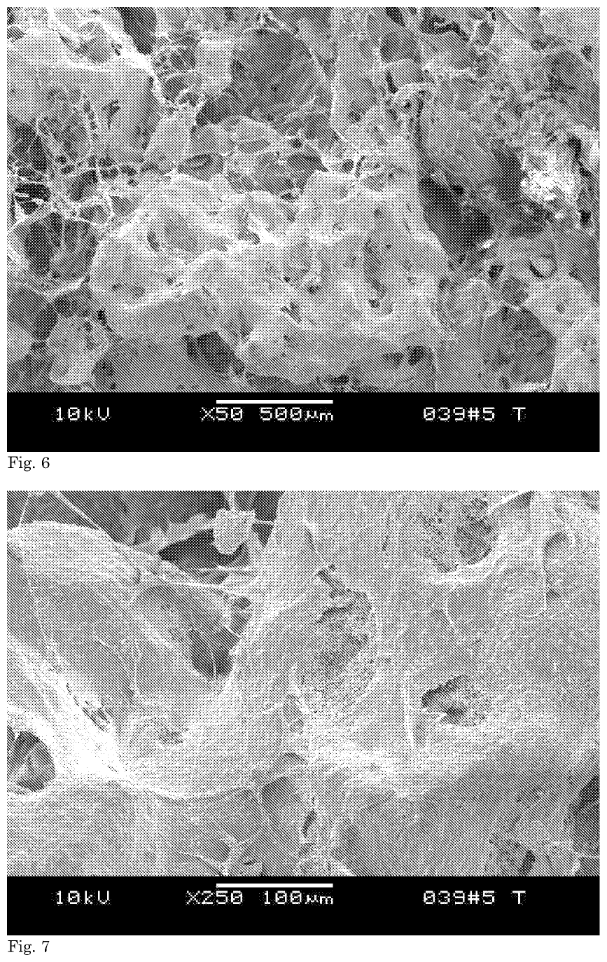

FIGS. 6 and 7 show SEM images of Comparative Example 2.

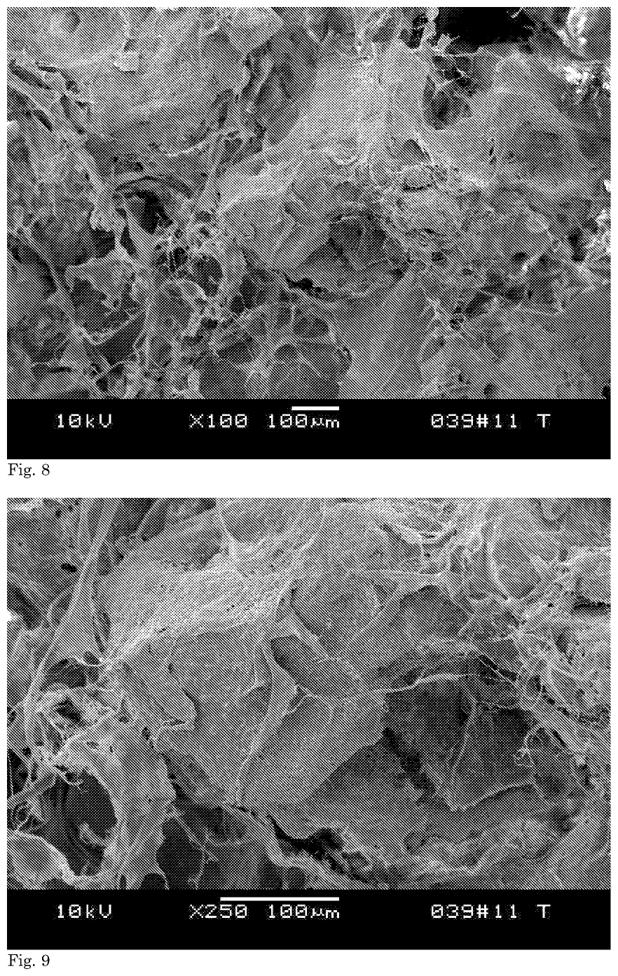

FIGS. 7 and 8 show SEM images of Comparative Example 10.

For comparison, i-Factor™, Formagraft™, Vitoss™ and Mastergraft™ constructs are also included.

Analytical Methods

Granular Exposure

The surface exposure of the granules in a composite were determined by SEM as follows. An SEM image from a top view of the composite was recorded. Then, for at least three representative granules on the SEM image, the surface of the granules was analyzed and the amount of said surface covered and exposed was measured using ImageJ (Schneider, C. A., Rasband, W. S., Eliceiri, K. W. “NIH Image to ImageJ: 25 years of image analysis” Nature Methods 9, 671-675, 2012).

A granule could be seen as a 3D Volume with angular lines and or circular pores representing irregularly shaped porous granules. When a surface of a granule is amorphous, smooth, shiny, homogeneous mass and/or unorganized with possible presence of cubical salts resulting from product processing, this surface was considered to be covered by matrix material. When a microsurface structure of a granule was visible on a surface, this surface was considered exposed.

Porosity

The porosity is expressed as the relative voids volume with respect to the total volume of the composite including said voids and was determined using the following equation:

Porosity %=1−(B_{d}/P_{d})

Wherein B_{d} is the bulk density of the composite determined by measuring the mass and volume, and P_{d} is the known particle density of the constituents in the ratio proportional to the composite's matrix:granule wt %.

Weight % Matrix/Granules

The weight ratio collagen versus granule was measured by burning away the organic material (i.e. collagen) using a furnace. After measuring the mass of the sample, it was placed in a crucible and into a furnace under the following conditions:
2 hrs to increase the heat from room temperature to 150° C.
7 hrs to increase the heat from 150° C. to 500° C.
3 hr 20 min to increase the heat from 500° C. to 600° C.
Hold at 600° C. for 3 hr 20 min
Furnace was shut off and slowly cooled back to room temperature overnight
Once cooled, the remaining inorganic material was weighed out, and the difference in weight of the material before and after placing it in the furnace was calculated using the following equation:

Ash Content %=M_f/M_i*100

where Mi is the initial material weight before placing it in the furnace and Mf is the final material weight after removing it from the furnace.

Morphology

An SEM image from the top view of a composite was recorded. Then, using ImageJ, the dimensions and content of fibers and sheets were measured and averaged. For this purpose, fibers were defined as structures having a thickness and width of less than 50 μm and sheets are defined as structure having a thickness and/or width of more than 50 μm.

The average fiber dimension was determined by using ImageJ to measure a minimum of 100 fibers and/or sheets per SEM image taken at a magnification of 250× or lower. The average was determined based off measurements from three representative SEM images.

In Vitro Testing

Wicking was determined by calculating the difference in wt % of a sample after immersion in a phosphate-buffered saline solution preheated to 37° C. for 20 sec. In this instance, the wicking ability is defined as the change in weight recorded between the dry weight and the hydrated weight.

Structure integrity after 12 days was determined by immersing a sample in a phosphate-buffered saline solution preheated to 37° C. for up to 12 days and determining if the sample remained structurally intact to the point where its dimensions could be accurately measured.

Mechanical Testing

Tensile Strength and elastic modulus was determined according to ISO 527-2-Plastics—Determination of Tensile Properties—Part 2: Test conditions for moulding and extrusion plastics.

TABLE 1

| Examples | | | Granular | | Morphology | | In vitro testing | | Mechanical | |
|---|---|---|---|---|---|---|---|---|---|---|
| (Ex.) and | | | exposure | | Fiber/ | Average | | Structure | testing | |
| Comparative | Weight % | | Surface | Exposed | sheet | fiber | | integrity | Tensile | Elastic |
| examples | matrix/ | Porosity | exposure | granules | content | dimensions | Wicking | after | Strength | Modulus |
| (Comp. ex.) | granules | (%) | (%) | (%) | (%) | (μm) | (wt %) | 12 days | (MPa) | (MPa) |
| Ex. 1 | 5.28/94.72 | 84 | 44.4 | 90 | 90/10 | 10.58 ± 14.64 | 141 ± 5 | yes | 0.25 ± 0.02 | 7.96 ± 0.21 |
| Comp. ex. 1 | 7.16/92.84 | 89 | 0 | 0 | 5/95 | | 208 | | | |
| Comp. ex. 2 | 4.44/95.56 | 86 | 0 | 0 | 15/85 | | 146 | | | |
| Comp. ex. 3 | 4.53/95.47 | 86 | 0 | 0 | 15/85 | | 169 | | | |
| Comp. ex. 4 | 10.11/89.89 | 87 | 0 | 0 | 60/40 | 22.34 ± 32.41 | 210 | | | |
| Comp. ex. 5 | 2.22/97.78 | | 1.1 | 20 | 15/85 | | —¹ | no | | |
| Comp. ex. 6 | 7.30/92.70 | 86 | 0 | 0 | 5/95 | | 212 | | | |
| Comp. ex. 7 | 4.39/95.61 | 86 | 0 | 0 | 5/95 | | 189 | | | |
| Comp. ex. 8 | 4.04/95.96 | | 0.9 | 13 | 25/75 | | —¹ | no | | |
| Comp. ex. 9 | 8.63/91.37 | 88 | 0 | 0 | 40/60 | | 269 | | | |
| Comp. ex. 10 | 2.48/97.52 | 85 | 1.6 | 17 | 25/75 | | 132 | | | |
| Vitoss ™ | 23.47/76.53 | 62 | 0 | 0 | 25/75 | | 14 | no | | |
| Forma-graft ™ | 10.14/89.85 | 88 | 5.44 | | 0/100 | | 262 | yes | | |
| Master-graft ™ | 2.72/97.28 | | 0 | | 5/95 | | 145 | yes | | |
| i-Factor | 14.872/85.13 | | 0 | | 0/100 | | —¹ | no | | |

¹desintegration
² weight % of silk-fiber matrix

In Vivo Study and Sample Evaluation

Four beagles (male, 12-month-old) were used and surgical operation was performed under general sterile conditions and anesthesia. MagnetOs Granules (controls) and most of the composites of Table 1 were intramuscularly implanted in back muscle (1 ml per sample), while the composite of Example 1 were also implanted in condyles ϕ6×10 mm) for collagen resorption analysis. Surgical operation was performed to get explants in different time points. At the end, the animals were sacrificed, and the samples were harvested with surrounding tissues. Routine un-decalcified histology was performed, sections (10-20 μm) were stained with methylene blue/basic fuchsin to view bone or van Gieson to view collagen. Histomorphometry was conducted with histological overviews and the area percentage of targets (e.g. collagen residue and bone) in the available space was calculated as target area*100/(region of interest-calcium phosphate (CaP) material).

One-way analysis of variance (ANOVA) with Tukey's post-test multiple comparisons and two-way ANOVA with Bonferroni's post-test multiple comparisons were performed.

In vivo stability of the composite was found to be as follows.

Collagen % for Intramuscular Implantation:
Week 0=10.2±1.7%
Week 3=3±0.4%
Week 6=0.2±0.3%
Week 12=0%

Collagen % for Femoral Condyle Implantation:
Week 0=10.2±1.7%
Week 3=1.4±1.7%
Week 6=0%
Week 12=0%

The bone growth results are shown in Table 2.

TABLE 2

| Examples and Comparative | 12 weeks intramuscular implantation |
|---|---|
| examples | Bone growth (%) |
| Example 1 | 13.5 |
| Comparative example 1 | <2.0% |
| Comparative example 2 | <<1.5% |
| Comparative example 3 | <2.5% |
| Comparative example 4 | <2.5 |
| Comparative example 5 | <2.75 |
| Comparative example 6 | <0.5 |
| Comparative example 7 | <1.5 |
| Comparative example 8 | <<0.75 |
| Comparative example 9 | <<0.5 |
| Comparative example 10 | <<0.75 |
| Vitoss ™ | 0 |
| Forma-graft ™ | Not tested |
| Master-graft ™ | Not tested |
| i-Factor | Not tested |

Statistical Analysis of Correlation Between Surface Exposure (%), Fiber Content (%) and Bone Formation (%)

A historical data design was constructed using Design Expert 13 (Build 13.0.1.0; Stat-Ease Inc.), using surface coverage (%) or fiber content (%) as continuous numerical factors and bone formation (%) as a response. A box-cox analysis revealed no data transformation was necessary when using surface coverage as the independent variable, lambda was maintained at 1.00. A linear regression model was generated and an ANOVA analysis found the effect from surface exposure was significant (<0.0001), with an R squared value of 0.935. When using fiber content as the independent variable, a box-cox analysis recommended a square-root transformation with a k value of 0.0135. The resulting linear regression model and ANOVA analysis found that the effect from fiber content was significant (0.0029) with an R squared value of 0.7279.

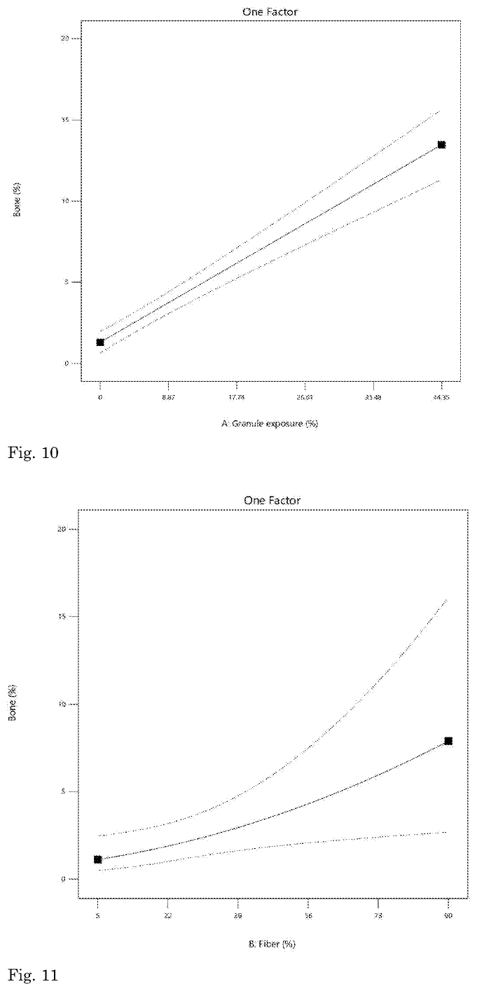

The result for surface exposure is provided in FIG. 10.

The result for fiber content is provided in FIG. 11.

1. A porous osteoinductive composite comprising osteoinductive granules comprised in a porous matrix, wherein more than 5% of the surface area of the osteoinductive granules is exposed from said matrix as determined by scanning electron microscopy (SEM) imaging.

2. The porous osteoinductive composite according to claim 1, wherein more than 10% of the surface area of the osteoinductive granules is exposed from said matrix as determined by scanning electron microscopy (SEM) imaging.

3. The porous osteoinductive composite according to claim 1, wherein more than 20% of the granules exhibit an at least partially exposed surface area as determined by scanning electron microscopy (SEM) imaging.

4. The porous osteoinductive composite according to claim 1 wherein said composite exhibits a porosity in the range of 60 to 95%.

5. The porous osteoinductive composite according to claim 1, wherein said matrix comprises fibers and sheets in a ratio of more than 1:1, wherein fibers are defined as structures having a thickness and width of less than 50 μm and sheets are defined as structure having a thickness and/or width of more than 50 μm.

6. The porous osteoinductive composite according to claim 1, wherein said matrix comprises fibers having an average diameter of less than 50 μm.

7. The porous osteoinductive composite according to claim 1, exhibiting wicking of more than 50 wt %.

8. The porous osteoinductive composite according to claim 1, wherein said matrix comprises pores and openings to said pores that are larger than mesenchymal stem cells and macrophages.

9. The porous osteoinductive composite according to claim 1, wherein the composite exhibits structural integrity for at least 5 days under phosphate-buffered saline solution at 37° C.

10. The porous osteoinductive composite according to claim 1, wherein the matrix comprises:
one or more of natural polymers selected from the group consisting of collagen, gelatin, fibrin, hyaluronic acid, silk fibroin, chitosan, alginate, cellulose, lignin, hydrogels derived from decellularized tissues, and other ECM-derived or ECM-mimicking natural polymers;
one or more semi-synthetic polymers;
one or more synthetic polymers selected from the group consisting of polyethylene glycol (PEG), polyoxamers, polylactic acid (PLA) poly(L-lactic) acid (PLLA), poly(ethylene glycol-co-lactic acid) (PELA), poly(poloxamer-co-lactic acid) (POLA), polyglycolic acid (PGA), poly(lactic-co-glycolic acid) (PGLA), polycaprolactone (PCL), polyvinyl alcohol (PVA), polyamides (PA), polyacrylonitrile (PAN), combinations and co-polymers thereof;
or combinations thereof.

11. The porous osteoinductive composite according to claim 1, wherein the matrix comprises collagen.

12. The porous osteoinductive composite according to claim 1, wherein the matrix comprises native collagen.

13. The porous osteoinductive composite according to claim 1, wherein the amount of the osteoinductive granules in the composite is more than 55 wt % and/or wherein the amount of the matrix in the composite is less than 45 wt % based on the weight of the composite.

14. The porous osteoinductive composite according to claim 1, wherein the osteoinductive granules comprise calcium phosphate.

15. The porous osteoinductive composite according to claim 1, wherein the composite comprises osteoinductive granules having a size in the range of 100 to 2500 μm.

16. The porous osteoinductive composite according to claim 1, exhibiting a tensile strength in the range of 0.1 MPa to 5 MPa and/or an elastic modulus in the range of 2 to 300 MPa.

17. The porous osteoinductive composite according to claim 1 in the form of a sheet, strip, block, rod or stick that is shapable such as pliable and/or flexible at 15° C.

18. A method for the preparation of the porous osteoinductive composite according to claim 1, said method comprising mixing the osteoinductive granules with a solution comprising the matrix, followed by lyophilizing said mixture.

19. The porous osteoinductive composite according to claim 1 for use in a medical treatment of connective tissue and/or bone loss or defect.

20. A method for the treatment of connective tissue and/or bone loss or defect of a patient, said method comprising providing the porous osteoinductive composite according to claim 1, optionally augmenting said composite with autologous tissue of said patient, and implanting the composite in the patient.
